(19) United States
(12) Patent Application Publication
(10) Pub. No.: US 2013/0210851 A1
(43) Pub. Date: Aug. 15, 2013

(54) Crystalline forms of a hexahydrofuro[3,4-c]quinoline derivative

(71) Applicants: Markus OSTERMEIER, Biberach an der Riss (DE); Kai GERLACH, Mittelbiberach (DE); Peter SIEGER, Mittelbiberach (DE)

(72) Inventors: Markus OSTERMEIER, Biberach an der Riss (DE); Kai GERLACH, Mittelbiberach (DE); Peter SIEGER, Mittelbiberach (DE)

(73) Assignee: Boehringer Ingelheim International GmbH, Ingelheim am Rhein (DE)

(21) Appl. No.: 13/587,982

(22) Filed: Aug. 17, 2012

(30) Foreign Application Priority Data
Aug. 18, 2011 (EP) 11178029.2

(51) Int. Cl. C07D 491/20; A61K 45/06; A61K 31/4741

(52) U.S. Cl. CPC C07D 491/20; A61K 31/4741; A61K 45/06
USPC 514/278; 546/15

(57) ABSTRACT

The present invention relates to crystalline forms of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol and its hydrochloride or hydrobromide salts, to a process for the manufacture thereof, and to the use thereof in pharmaceutical compositions.

The present invention relates to hydrochloride and hydrobromide salts of a hexahydrofuro[3,4-c]quinoline derivative, namely crystalline hydrochloride and hydrobromide salts of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, as well as to crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, to a process for the manufacture thereof, and to the use thereof in pharmaceutical compositions.

International Patent Application PCT/EP2011052376 discloses hexahydrofuro[3,4-c]quinoline derivatives, including (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, the structure of which compound is depicted below in the form of the free base as formula (I), a process for their manufacture and their use in a pharmaceutical composition to treat and/or prevent disorders which can be influenced by inhibiting cholesteryl ester transfer protein (CETP), such as e.g. cardiometabolic or cardiovascular disorders.

Although the pharmacologically valuable properties of the hexahydrofuro[3,4-c]quinoline derivatives disclosed in the art and mentioned above constitute the basic prerequisite for effective use of the compounds as medicaments or in pharmaceutical compositions, an active substance must in any case satisfy additional requirements in order to be accepted for use as a drug or in a pharmaceutical dosage form. These parameters are largely connected with the physicochemical nature of the active substance, particularly in its solid form.

Hence, there continues to be a need for novel crystalline forms of active substances, which can be conveniently formulated for administration to patients and which are substantially pure and highly crystalline in order to fulfil exacting pharmaceutical requirements and specifications.

Preferably, such compounds will be readily formed in suitable yields, exhibit good upscale ability, manufacturability and processability and have favourable bulk characteristics. Examples of favourable bulk characteristics are drying times, bulk density, flowability, filterability, solubility, intrinsic dissolution rate, stability in general and especially thermal stability, and hygroscopicity.

An absence of breakdown products in the pharmaceutical composition being used is also favourable, since if breakdown products are present in the pharmaceutical composition the content of active substance present in the pharmaceutical formulation might be lower than specified.

Another critical parameter to be controlled is the hygroscopicity, since the absorption of moisture reduces the content of pharmaceutically active substance as a result of the increased weight caused by the uptake of water. Pharmaceutical compositions with a tendency to absorb moisture have to be protected from moisture during storage, e.g. by the addition of suitable drying agents or by storing the drug in an environment where it is protected from moisture. In addition, the uptake of moisture may reduce the content of pharmaceutically active substance during manufacture if the pharmaceutical substance is exposed to the environment without being protected from moisture in any way. Preferably, therefore, the hygroscopicity of a pharmaceutically active substance should be well characterised, and possibly also stabilized.

As the crystal modification of an active substance is important to the reproducible active substance content of a preparation, there is a need to clarify as far as possible any existing polymorphism of an active substance present in crystalline form. If there are different polymorphic modifications of an active substance care must be taken to ensure that the crystalline modification of the substance does not change in the pharmaceutical preparation later produced from it. Otherwise, this could have a harmful effect on the reproducible potency of the drug. Against this background, active substances characterised by only slight polymorphism are preferred.

Another criterion which may be of importance under certain circumstances depending on the choice of formulation or the choice of manufacturing process is the solubility and dissolution behaviour of the active substance. If for example pharmaceutical solutions are prepared (e.g. for infusions) it is essential that the active substance should be sufficiently soluble in physiologically acceptable solvents, particularly aqueous media. For drugs which are to be taken orally, it is in general very important that the active substance should be sufficiently soluble, readily dissolvable and bioavailable.

Decreased levels of organic solvents in the crystal lattice are also favourable, due in part to potential solvent toxicity to the recipient as a function of the solvent.

Under certain circumstances, it may be also favourable for drug development to use an anhydrous form than a hydrate form, since, for example, preparation and handling of hydrates might be sometimes difficult as reproducibility and stability of the hydrated forms may depend on external influences in complex manner, or some hydrates might tend to be less soluble with respect to homologous anhydrous forms, with potential detrimental effect also on the dissolution rate properties of the active compound per se and on its absorption profile through the gastrointestinal tract.

Furthermore, the process for preparing such a compound also needs to be conveniently carried out on commercial scale.

Hence, without being restrictive, examples of the parameters which need to be controlled are the (stress) stability of the starting substance under various environmental conditions, the stability during production of the pharmaceutical formulation and the stability in the final compositions of the drug.

The pharmaceutically active substance used to prepare the pharmaceutical compositions should therefore have great (chemical and physical) stability which is ensured even under all kinds of environmental conditions.

Moreover, as it may be of further advantage for acceptance for use as a drug, it may be favourable that the active substance is suitable for oral administration. Likewise, it may be favourable that the active substance is useful for the manufacture of solid oral pharmaceutical forms, such as tablets and capsules, or liquid oral pharmaceutical forms, such as orally administered solutions and suspensions, whereby emphasis might be given to solid oral dosage forms.

Typically, in the preparation of a pharmaceutical composition, a form of the active ingredient is sought that has a balance of desired properties. Therefore it is desired to provide a pharmaceutically active substance which is not only characterised by high pharmacological potency but also satisfies the above-mentioned physicochemical requirements as far as possible.

Thus, an aim of the invention is to provide new compounds of formula (I), particularly salts with interesting and useful properties suitable for pharmaceutical use.

Other aims of the present invention may become apparent to the skilled man directly from the foregoing and following remarks.

### SUMMARY OF THE INVENTION

It has been found that (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol, the structure of which compound is depicted below in the form of the free base as formula (I), can be prepared in crystalline form and also in the form of a hydrochloride or hydrobromide salt thereof, and preferably a crystalline hydrochloride or hydrobromide salt form, more preferably a crystalline hydrochloride salt form.

The present invention provides the compound of formula (I) in crystalline form and also in the form of a crystalline hydrochloride or hydrobromide salt of the compound of formula (I) (i.e. the crystalline forms according to this invention), each form described in greater details herein.

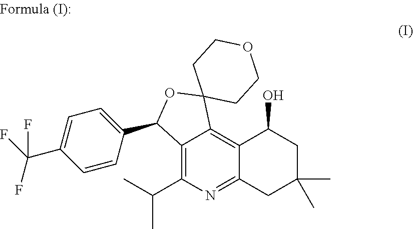 [SMILES: CC(C)c1nc2c(c3c1[C@@H](c1ccc(C(F)(F)F)cc1)OC31CCOCC1)[C@@H](O)CC(C)(C)C2]

Moreover, it has been further found that the problem outlined above is solved by the crystalline hydrochloride salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol and that this crystalline hydrochloride salt form has suitable solid state properties and is particularly suitable for the purposes of this invention.

Further, it has been found that the crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol has also satisfying solid state properties.

Yet further, it has been found that the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol has also satisfying solid state properties.

Hence, the crystalline hydrochloride and hydrobromide salt forms of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol and the crystalline free base according to this invention, each described in greater details herein, have interesting and useful properties.

For example the crystalline forms according to this invention can be formulated in pharmaceutical dosage forms, particularly solid oral pharmaceutical dosage forms such as tablets or capsules.

The crystalline forms according to this invention are non-hygroscopic anhydrous forms, which are highly crystalline and are stable in the solid state even under harsh stress conditions.

The crystalline hydrochloride and hydrobromide salt forms of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention has added properties, such as e.g. improved solubility, high bulk density, good flowability, and no indications for polymorphism for both salts in contrast to the crystalline free base, which exhibits enantiotropic polymorphism.

The crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention has further added properties, such as e.g. high drug load in tablet and high yield of salt formation. This crystalline hydrochloride form is a non-hygroscopic anhydrous form (e.g. uptake of only ca. 0.4% of water up to 90 r.h.), highly crystalline and exhibits good solubility up to pH 4.0 and is stable in the solid state. In contrast, the amorphous hydrochloride form is hygroscopic and is capable to take up significant amounts of water (e.g. uptake of ca. 9% of water up to 90% r.h.).

The following terms are used synonymously:

salt with hydrochloric acid—hydrochloride;
salt with hydrobromic acid—hydrobromide.

Accordingly, the compounds according to this invention as provided and referred to herein particularly relate to the crystalline HCl or HBr salt or free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol (compound of formula (I)), each as described herein.

Thus, in a broadest embodiment, the present invention relates to crystalline acid addition salts of the compound of formula (I) with hydrochloric or hydrobromic acid, as well as to crystalline compound of formula (I), all of them referred to herein as the crystalline forms according to this invention.

In a preferred embodiment, the present invention relates to the hydrochloride salt of the compound of formula (I),

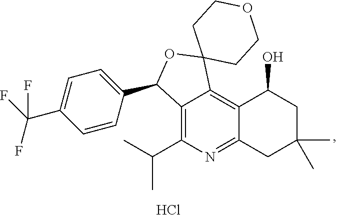 [SMILES: CC(C)c1nc2c(c3c1[C@@H](c1ccc(C(F)(F)F)cc1)OC31CCOCC1)[C@@H](O)CC(C)(C)C2 ; Cl]

particularly a crystalline form thereof especially as described herein.

Accordingly, a first aspect of the present invention refers to a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

Preferably, the hydrochloride salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to the invention is anhydrous and non-solvated, and contains about 1 molar equivalent of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol per molar equivalent of HCl, i.e. it is the monohydrochloride salt.

A further object of the present invention refers to a process as well as intermediates for making a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

A further object of the present invention refers to a pharmaceutical composition comprising a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, together with one or more pharmaceutically acceptable carriers, diluents or excipients.

A further object of the present invention refers to a method for treating and/or preventing disorders which can be influenced by inhibiting the enzyme cholesteryl ester transfer protein (CETP), such as e.g. cardiometabolic or cardiovascular disorders, comprising administering an effective amount of a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol to a patient (particularly human patient) in need thereof.

A further object of the present invention refers to a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for use in a method for treating and/or preventing disorders which can be influenced by inhibiting cholesteryl ester transfer protein (CETP), such as e.g. cardiometabolic or cardiovascular disorders.

A further object of the present invention refers to the use of a crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for preparing a pharmaceutical composition which is suitable for treating and/or preventing disorders which can be influenced by inhibiting cholesteryl ester transfer protein (CETP), such as e.g. cardiometabolic or cardiovascular disorders.

A further object of the present invention refers to a quantity of the compound of formula (I) wherein at least 50%, preferably at least 75%, more preferably at least 95%, even more preferably at least 99%, of said substance is present in the form of a crystalline hydrochloride salt of the compound of formula (I) as defined herein, e.g. as may be characterized by any of the hereinmentioned XRPD-defined embodiments. The presence of such amounts of crystalline hydrochloride salt of the compound of formula (I) is typically measured using XRPD analysis of the compound.

A further object of the present invention refers to a pharmaceutical composition comprising a hydrochloride salt of the compound of formula (I) and optionally one or more pharmaceutically acceptable carriers and/or diluents, wherein at least 50%, preferably at least 75%, more preferably at least 95%, even more preferably at least 99%, of said active substance is present in crystalline form, for example in the form of a crystalline hydrochloride salt of the compound of formula (I), e.g. as may be characterized by any of the hereinmentioned XRPD-defined embodiments.

A second aspect of the present invention refers to a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

Preferably, the hydrobromide salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to the invention is anhydrous and non-solvated, and contains about 1 molar equivalent of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol per molar equivalent of HBr, i.e. it is the monohydrobromide salt.

A further object of the present invention refers to a process as well as intermediates for making a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

A further object of the present invention refers to a pharmaceutical composition comprising a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, together with one or more pharmaceutically acceptable carriers, diluents or excipients.

A further object of the present invention refers to a method for treating and/or preventing disorders which can be influenced by inhibiting the enzyme cholesteryl ester transfer protein (CETP), such as e.g. cardiometabolic or cardiovascular disorders, comprising administering an effective amount of a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol to a patient (particularly human patient) in need thereof.

A further object of the present invention refers to a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for use in a method for treating and/or preventing disorders which can be influenced by inhibiting cholesteryl ester transfer protein (CETP), such as e.g. cardiometabolic or cardiovascular disorders.

A further object of the present invention refers to the use of a crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for preparing a pharmaceutical composition which is suitable for treating and/or preventing disorders which can be influenced by inhibiting cholesteryl ester transfer protein (CETP), such as e.g. cardiometabolic or cardiovascular disorders.

A further object of the present invention refers to a quantity of the compound of formula (I) wherein at least 50%, preferably at least 75%, more preferably at least 95%, even more preferably at least 99%, of said substance is present in the form of a crystalline hydrobromide salt of the compound of formula (I) as defined herein.

A further object of the present invention refers to a pharmaceutical composition comprising a hydrobromide salt of the compound of formula (I) and optionally one or more pharmaceutically acceptable carriers and/or diluents, wherein at least 50%, preferably at least 75%, more preferably at least 95%, even more preferably at least 99%, of said active substance is present in crystalline form, for example in the form of a crystalline hydrobromide salt of the compound of formula (I).

A third aspect of the present invention refers to a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

A further object of the present invention refers to a process as well as intermediates for making a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

A further object of the present invention refers to a pharmaceutical composition comprising a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, together with one or more pharmaceutically acceptable carriers, diluents or excipients.

A further object of the present invention refers to a method for treating and/or preventing disorders which can be influenced by inhibiting the enzyme cholesteryl ester transfer protein (CETP), such as e.g. cardiometabolic or cardiovascular disorders, comprising administering an effective amount of a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol to a patient (particularly human patient) in need thereof.

A further object of the present invention refers to a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for use in a method for treating and/or preventing disorders which can be influenced by inhibiting cholesteryl ester transfer protein (CETP), such as e.g. cardiometabolic or cardiovascular disorders.

A further object of the present invention refers to the use of a crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for preparing a pharmaceutical composition which is suitable for treating and/or preventing disorders which can be influenced by inhibiting cholesteryl ester transfer protein (CETP), such as e.g. cardiometabolic or cardiovascular disorders.

A further object of the present invention refers to a quantity of the compound of formula (I) wherein at least 50%, preferably at least 75%, more preferably at least 95%, even more preferably at least 99%, of said substance is present in the form of a crystalline free base form of the compound of formula (I) as defined herein.

A further object of the present invention refers to a pharmaceutical composition comprising a crystalline free base form of the compound of formula (I) and optionally one or more pharmaceutically acceptable carriers and/or diluents, wherein at least 50%, preferably at least 75%, more preferably at least 95%, even more preferably at least 99%, of said active substance is present in crystalline form, for example in the form of a crystalline free base form of the compound of formula (I).

This invention also relates to a crystalline form according to the present invention which is suitable for inhibiting cholesteryl ester transfer protein (CETP).

The invention also relates to a process for preparing a pharmaceutical composition according to the invention, comprising incorporating at least one crystalline form according to the invention in one or more pharmaceutically acceptable carriers and/or diluents preferably by a non-chemical method.

The present invention also relates to a pharmaceutical composition comprising or made from a therapeutically effective amount of at least one crystalline form according to the invention, and optionally one or more pharmaceutically acceptable carriers and/or diluents.

The present invention also relates to a method for treating and/or preventing a disease or condition which can be influenced by inhibiting cholesteryl ester transfer protein (CETP), e.g. a cardiovascular, cardiometabolic or related disorder, such as e.g. any of those diseases and conditions mentioned herein, in a mammalian (particularly human) patient in need thereof comprising administering to said patient a therapeutically effective amount of at least one crystalline form according to the invention.

The present invention also relates to a crystalline form according to this invention for use in a method of treating and/or preventing a condition which can be influenced by inhibiting cholesteryl ester transfer protein (CETP), e.g. a cardiovascular, cardiometabolic or related disorder, such as e.g. any of those diseases and conditions mentioned herein, said method comprising administration of said crystalline form, optionally alone or in combination (such as e.g. separately, sequentially, simultaneously, concurrently or chronologically staggered) with one or more other therapeutic agents, such as e.g. selected from those mentioned herein.

The present invention also relates to a crystalline form according to this invention for use in a method of treating and/or preventing a cardiovascular, cardiometabolic or related disorder selected from

atherosclerosis, dyslipidemia (e.g. mixed dyslipidemia), hyperbeta-lipoproteinemia, hypoalpha-lipoproteinemia, hypercholesterolemia, hypertriglyceridemia, hyperlipidemia, hypolipoproteinemia, hyperlipoproteinemia, hypo-HDL cholesterolemia, hyper-LDL cholesterolemia, familial hypercholesterolemia, peripheral vascular disease, hypertension, endothelial dysfunction, angina, ischemia, cardiac ischemia, stroke, myocardial infarction, reperfusion injury, angioplastic restenosis, arteriosclerosis, coronary heart disease, coronary artery disease, coronary vascular disease or congestive heart failure, vascular complications of diabetes, insulin resistance, obesity, metabolic syndrome, diabetes (especially type 2 diabetes mellitus) or endotoxemia, said method comprising administration of said crystalline form, optionally in monotherapy or in combination therapy (such as e.g. separately, sequentially, simultaneously, concurrently or chronologically staggered) with one or more other therapeutic agents, such as e.g. selected from those mentioned herein, such as e.g. a HMG-CoA reductase inhibitor (e.g. a statin).

The present invention also relates to a crystalline form according to this invention for use in a method of increasing patient's levels of HDL cholesterol and/or decreasing patient's levels of VLDL cholesterol and/or of LDL cholesterol, optionally in combination with one or more other therapeutic agents, such as e.g. selected from those mentioned herein, such as e.g. a HMG-CoA reductase inhibitor (e.g. a statin).

The present invention also relates to a crystalline form according to this invention for use in a method of primary or secondary prevention of cardiovascular diseases, particularly major cardiovascular events, optionally in combination with one or more other therapeutic agents, such as e.g. selected from those mentioned herein, such as e.g. a HMG-CoA reductase inhibitor (e.g. a statin).

In certain embodiments, the present invention also relates to the crystalline forms according to the present invention which are in substantially pure form (e.g. substantially devoid of impurities and/or other forms), for example in a degree of purity of about of about >80%, >85%, >90%, >95%, >98%, or >99% of the respective form.

In certain embodiments, the present invention also relates to crystalline forms according to the present invention in substantially pure form, that means, for example, that the respective form includes less than 20%, less than 10%, less than 5%, less than 3% or less than 1% by weight of any impurities or other physical forms.

Other aspects of the present invention will become apparent from the description hereinbefore and hereinafter (including the examples) as well as the claims.

### BRIEF DESCRIPTION OF THE FIGURES

FIG. 1 shows the X-ray powder diffractogram of the crystalline anhydrous hydrochloride salt of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol according to this invention, recorded using a STOE STADI P diffractometer in transmission fitted with a position-sensitive detector (PSD) and a Cu anode as the X-ray source with monochromated CuK_{α1} radiation (λ=1.54056 Å, 40 kV, 40 mA).

FIG. 2 shows the thermoanalysis (DSC/TG) of the crystalline anhydrous hydrochloride salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention, the DSC/TG data are collected on a Mettler DSC822e/TGA/SDTA851e system.

### DETAILED DESCRIPTION OF THE INVENTION

Abbreviations used:
TLC Thin-Layer Chromatography
DSC Differential Scanning calorimeter
TG ThermoGravimetry
XRPD X-ray powder diffraction

Crystalline hydrochloride salt of the compound of formula (I):

The following solid state characteristics, solubility, dissolution, stability and preparation of the crystalline hydrochloride salt of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol are typically relevant to the present invention.

Thus, the present invention relates to the crystalline hydrochloride salt form (particularly crystalline monohydrochloride salt form) of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol, as may be characterized by one or more of the following characteristics.

Preparation of the Crystalline (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol Hydrochloride According to this Invention

Generally speaking the crystalline hydrochloride salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol is prepared by combining (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol (the free base of formula (I)) preferably dissolved or suspended in a suitable solvent and HCl (e.g. used as HCl in dioxane, HCl in diethylether, HCl in tert.-butylmethylether, gaseous HCl, hydrochloric acid, or the like) at a suitable temperature (e.g. room temperature or elevated temperature) to provide the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention, which is isolated and, optionally, purified, washed and/or dried.

Examples of such suitable solvents may include organic solvents such as ketones, e.g. acetone, methyl ethyl ketone or methyl isobutyl ketone, or ethers, e.g. diethyl ether, diisopropyl ether or tert.-butylmethylether, or the like, or mixtures thereof; with ketones and, especially, acetone being preferred.

In more detail, the present invention provides a method of making the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol which comprises:

Contacting a solution of the compound of formula (I) (the free base) with HCl to form the hydrochloride salt of the compound of formula (I).

In certain embodiments, the solution of the compound of formula (I) comprises acetone. In other certain embodiments, the HCl is used as HCl in dioxane.

In a particular embodiment, the reaction is performed using reactants and/or one or more solvents that are substantially free of water (e.g. almost completely free of water).

In a further embodiment, the method further comprises crystallizing the crystalline hydrochloride salt form of the compound of formula (I) from the reaction solution. In certain embodiments, the crystals are precipitated, e.g. either spontaneously or being induced (e.g. by cooling, adding a non-solvent, concentration of the solvent(s) and/or by (azeotropic) removal of water, if present; such as e.g. by codistillation of the acetone/water azeotrope).

In a yet further embodiment, the method further comprises isolating or collecting the crystals of the crystalline hydrochloride salt form of the compound of formula (I). In certain embodiments, the crystals are isolated by filtration.

In a still yet further embodiment, the method further comprises optionally washing and/or drying the isolated crystalline hydrochloride salt form of the compound of formula (I). In certain embodiments, the crystals are dried at a suitable temperature, e.g. at a temperature of about 30° C. In other certain embodiments, the crystals are dried under reduced pressure. The drying step may be conducted for a suitable period of time.

Solid State Characteristics of the Crystalline (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol Hydrochloride According to this Invention

Crystallinity and Polymorphism

This crystal form of the hydrochloride salt of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol is highly crystalline.

In an embodiment, the material appears as white microcrystalline powder (plate- or rod-like shape). Under polarized light microscope rod-like crystals are observed which tend to agglomerate and show clear birefringence.

Bulk properties are favorable with high bulk density and good flowability. So far there are no indications for polymorphism for this hydrochloride salt form.

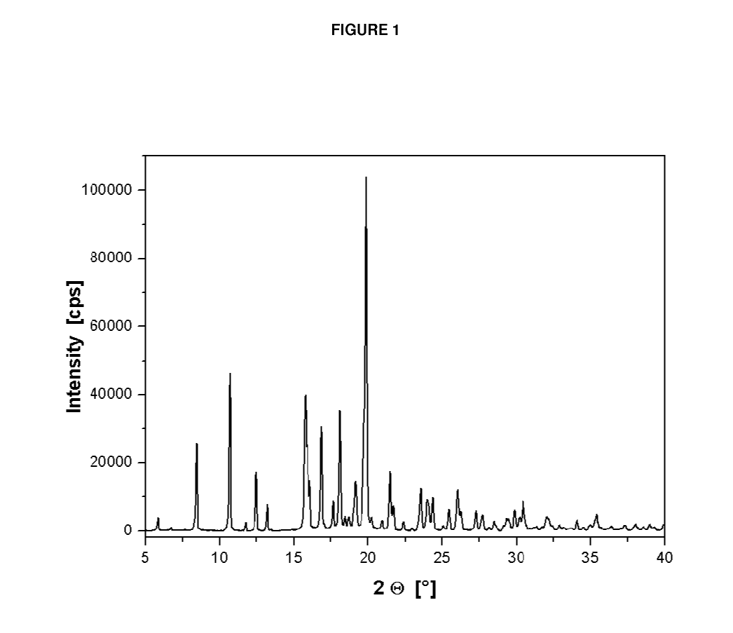

The X-ray powder diffraction diagram of this (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol hydrochloride is shown in FIG. 1.

The related X-ray powder reflections/indexed XRPD peaks up to 30° 2 0 and intensities (normalized) of this (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol hydrochloride are shown in the following Table 1 (wavelength: λ=1.54056 Å).

TABLE 1

| 2Θ | dₕₖₗ | Intensity | Indexing | | | 2Θ_{obs} − 2Θ_{calc} |
|---|---|---|---|---|---|---|
| [°] | [Å] | I/Iₒ [%] | h | k | l | [°] |
| 5.86 | 15.07 | 5 | 2 | 0 | 0 | −0.002 |
| 6.68 | 13.23 | 2 | 1 | 1 | 0 | −0.03 |
| 8.42 | 10.50 | 31 | 2 | 1 | 0 | 0.001 |
| 10.67 | 8.28 | 43 | 3 | 1 | 0 | −0.001 |
| 11.73 | 7.54 | 3 | 4 | 0 | 0 | −0.01 |
| 12.44 | 7.11 | 20 | 1 | 2 | 0 | 0.003 |
| 13.21 | 6.70 | 7 | 4 | 1 | 0 | <0.001 |
| 15.79 | 5.61 | 42 | 0 | 1 | 1 | −0.008 |
| 16.08 | 5.51 | 11 | 1 | 1 | 1 | 0.006 |
| 16.87 | 5.25 | 29 | 2 | 1 | 1 | 0.01 |
| 17.66 | 5.02 | 6 | 6 | 0 | 0 | 0.017 |
| 18.12 | 4.89 | 31 | 3 | 1 | 1 | 0.011 |
| 18.42 | 4.81 | 4 | 1 | 3 | 0 | 0.012 |
| 18.73 | 4.73 | 3 | 4 | 0 | 1 | −0.036 |
| 19.12 | 4.64 | 13 | 2 | 3 | 0 | 0.019 |
| 19.89 | 4.46 | 100 | 2 | 2 | 1 | 0.01 |
| 20.22 | 4.39 | 4 | 3 | 3 | 0 | 0.007 |
| 20.97 | 4.23 | 3 | 3 | 2 | 1 | 0.015 |
| 21.49 | 4.13 | 12 | 6 | 2 | 0 | 0.043 |
| 21.70 | 4.09 | 7 | 4 | 3 | 0 | 0.017 |
| 22.39 | 3.97 | 3 | 4 | 2 | 1 | 0.014 |
| 22.98 | 3.87 | 1 | 6 | 0 | 1 | 0.012 |
| 23.57 | 3.77 | 12 | 1 | 3 | 1 | 0.006 |
| 23.99 | 3.71 | 6 | 7 | 2 | 0 | 0.022 |
| 24.10 | 3.69 | 6 | 5 | 2 | 1 | 0.019 |
| 24.37 | 3.65 | 6 | 8 | 1 | 0 | −0.01 |
| 25.03 | 3.56 | 1 | 3 | 3 | 1 | 0.008 |
| 25.44 | 3.50 | 7 | 6 | 3 | 0 | 0.004 |
| 26.03 | 3.42 | 10 | 6 | 2 | 1 | <0.001 |
| 26.23 | 3.39 | 5 | 4 | 3 | 1 | <0.001 |
| 27.30 | 3.26 | 5 | 9 | 1 | 0 | −0.01 |
| 27.70 | 3.22 | 4 | 5 | 3 | 1 | −0.017 |
| 28.16 | 3.17 | 1 | 7 | 2 | 1 | −0.003 |
| 28.47 | 3.13 | 3 | 5 | 4 | 0 | −0.047 |
| 28.62 | 3.12 | 2 | 1 | 4 | 1 | 0.007 |
| 29.08 | 3.07 | 2 | 2 | 4 | 1 | −0.003 |
| 29.32 | 3.04 | 3 | 9 | 2 | 0 | 0.008 |
| 29.44 | 3.03 | 3 | 0 | 0 | 2 | 0.023 |
| 29.83 | 2.99 | 8 | 3 | 4 | 1 | −0.008 |
| 30.19 | 2.96 | 5 | 6 | 4 | 0 | −0.003 |
| 30.46 | 2.93 | 8 | 8 | 2 | 1 | −0.001 |

X-ray powder diagrams are generated using a STOE-STADI P-diffractometer in transmission mode fitted with a position-sensitive detector (PSD) and a Cu-anode as X-ray source with monochromated CuK_{α1} radiation. (λ=1.54056 Å, 40 kV, 40 mA)

In Table 1 above the value “2 Θ [°]” denotes the angle of diffraction in degrees and the value “dₕₖₗ [Å]” denotes the specified distances in Å between the lattice planes.

Lattice metrics of this crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol are as follows:

Indexing is possible with an orthorhombic cell with the following cell constants: a=30.13(1) Å, b=14.64(1) Å, c=6.068(4) Å, α=β=γ=90°, V=2677(3) Å³. All 41 reflexes can be indexed with a figure of merit of 53.7. According to the extinction conditions space group Pna2₁ (#33) can be assigned.

Accordingly, the present invention relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having a x-ray diffraction pattern substantially in accordance with that shown in FIG. 1.

In a further embodiment, the present invention relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, characterised by unit cell parameters approximately equal to the following:

Cell dimensions:
a=30.13(1) Å,
b=14.64(1) Å,
c=6.068(4) Å,
α=β=γ=90°,

### Volume=2677(3) Å³,

Space group Pna2₁.

The present invention further relates to the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol having a XRPD pattern comprising one or more of the following: a peak at 19.89, 10.67, 15.79, 18.12, 8.42, 16.87, 12.44, 19.12, 21.49, 23.57, 16.08 and 26.03 degrees 20 (e.g. each about ±0.05-0.3 degrees 2θ).

The present invention further relates to the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol, characterised in that in the x-ray powder diagram it has, inter alia, the characteristic values d=4.46 Å, 4.89 Å, 5.25 Å, 5.61 Å, 8.28 Å and 10.50 Å (e.g. most prominent peaks in the diagram with an intensity of more than about 30%).

Further, according to the findings shown in Table 3 the present invention further relates to the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol, characterised in that in the x-ray powder diagram it has, inter alia, the characteristic values d=4.46 Å, 4.89 Å, 5.25 Å, 5.61 Å, 7.11 Å, 8.28 Å and 10.50 Å, e.g. with an intensity of more than about 20%.

Further on, according to the findings shown in Table 3 the present invention further relates to the crystalline anhydrous hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, characterised in that in the x-ray powder diagram it has, inter alia, the characteristic values d=3.42 Å, 3.77 Å, 4.13 Å, 4.46 Å, 4.64 Å, 4.89 Å, 5.25 Å, 5.51 Å, 5.61 Å, 7.11 Å, 8.28 Å and 10.50 Å, e.g. with an intensity of more than about 10%.

Under normal conditions the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to the invention is present in an ansolvate and/or anhydrous (non-hydrate) form.

To study the hygroscopic behaviour of this material, sorption isotherms are registered on a DVS-1 water sorption monitor from Surface Measurement Systems. Adsorption and desorption isotherms are performed at 25° C. with 10% r.h. step intervals ranging from 10% r.h. up to 90% r.h.

It is found that the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol according to the invention is not hygroscopic. Up to 90% r.h. an uptake of only about 0.4% of water is observed which is fully reversible. No solid phase change is observed during moisture sorption/desorption.

Accordingly, in an embodiment, the present invention further relates to the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol, characterised in that it is an anhydrous form.

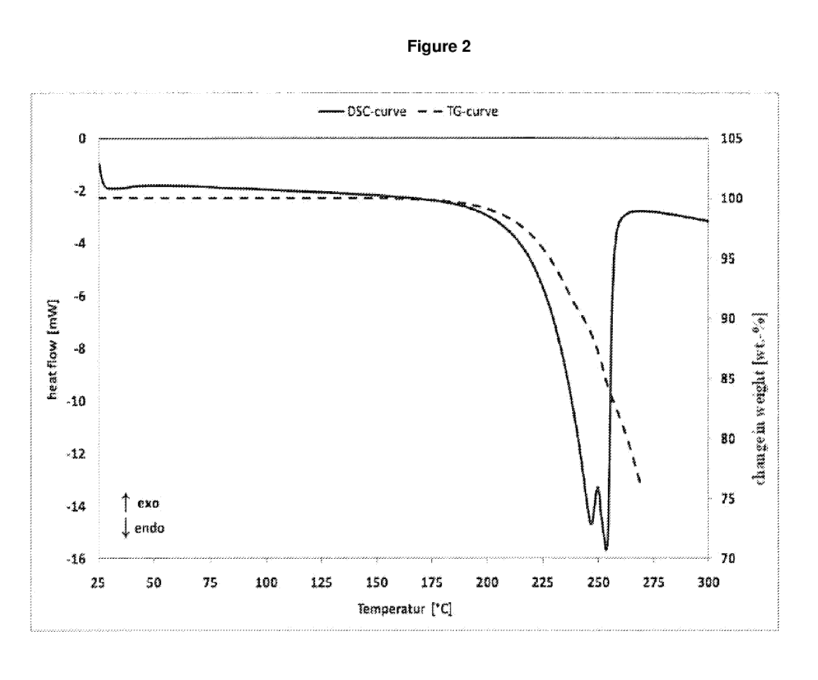

The thermoanalysis of the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol according to the invention shows only one broad endothermic event between about 200-250° C. (DSC: 10 K·min⁻¹ heating rate) (T_{fus}=approximately 220±10° C.). This event can be attributed to melting and thermal decomposition of the compound. The release of HCl can be detected above 200° C. in corresponding TG-IR coupling experiments indicating incongruent melting of the compound. Before the incongruent melting event no other signals can be detected in the DSC-diagram and substantially no weight loss in the corresponding TG-diagram is observed (loss of drying typically <0.5% release of water as shown by TG-IR coupling), indicating the anhydrous nature of this form. The DSC/TG diagram is shown in FIG. 2. DSC/TG data are collected on a Mettler DSC822e/TGA/SDTA851e system.

Accordingly, the present invention relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having a DSC and/or TG thermal curve substantially in accordance with that shown in FIG. 2 at a heating rate of 10 K per minute.

In a further embodiment, the present invention further relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having a fusion temperature of T_{fus}=about 220±10° C. (determined by DSC; heating rate: 10 K/min).

The present invention further relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, characterised in that its DSC profile shows a broad endothermic event between about 200-250° C., which may be attributed to melting and thermal composition of the compound.

The present invention further relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, characterised in that its TG profile shows significant mass loss above about 200° C., which may attributed to release of HCl.

In a further embodiment, the present invention relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having a x-ray diffraction pattern substantially in accordance with that shown in FIG. 1 and a DSC thermal curve substantially in accordance with that shown in FIG. 2 at a heating rate of 10 K per minute.

In a further embodiment, the present invention relates to the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having at least one characteristic of any of the hereinmentioned XRPD-defined embodiments and at least one characteristic of any of the hereinmentioned DSC/TG-defined embodiments.

Solid State Stability of the Crystalline (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol Hydrochloride According to this Invention

When testing the stability of the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol under harsh stress conditions for solid state (24 hours at 105° C., 72 hours at 60° C. and 70% rel. humid., or 24 hours in a Xenotester (A=300-800 nm, 250 W/m²)), the results show that the hydrochloride salt of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol is stable in the solid state. Under all applied severe stress conditions only very low or minor decomposition (Σ</=about 6% degradation impurities measured as peak area percentage from HPLC-diagrams using a standard gradient technique) can be observed.

Solubility and dissolution properties of the crystalline (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol hydrochloride according to this invention:

Solubility in Aqueous Media

Table 2 shows the values of solubility (mg/ml) of the saturated solution of this crystalline (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol hydrochloride in different aqueous media after 48 h vigorous shaking at room temperature.

TABLE 2

| | solubility [mg/ml] | |
|---|---|---|
| medium | RT - values | intrinsic pH |
| water | 3.1 | 1.6 |
| 0.1N HCl | 4.1 | 0.9 |
| 0.01N HCl | 1.9 | 1.9 |
| McIlvaine pH 2.2 | 1.6 | 2.0 |
| McIlvaine pH 3.0 | 0.4 | 3.0 |
| Acetate pH 3.0 | 0.2 | 3.0 |
| McIlvaine pH 4.0 | 0.09 | 4.0 |
| Acetate pH 4.0 | 0.06 | 4.2 |
| McIlvaine pH 4.5 | 0.03 | 4.6 |
| McIlvaine pH 5.0 | 0.003 | 5.1 |
| Acetate pH 5.0 | 0.006 | 5.3 |
| McIlvaine pH 6.0 | <0.003 | 6.2 |
| McIlvaine pH 6.8 | <0.003 | 7.0 |
| McIlvaine pH 7.4 | 0.00026^{#} | 7.7 |
| Sorensen pH 10.0 | <0.003 | 9.6 |
| FaSSIF * | 0.02 | 6.5 |
| FeSSIF * | 0.03 | 5.0 |

* FaSSiF & FeSSIF = Fasted and Fed State Simulated Intestinal
^{#}determined by LC/MS

From the above results, it can be concluded that the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol has a pH-dependent solubility profile in aqueous media with a good solubility up to pH 4.0 and poor solubility above pH 4.0 due to the lower solubility of the free base. A significant increase of solubility is observed in simulated intestinal fluids where bile acids could have an influence on the solubility.

Intrinsic Dissolution Rate in Aqueous Media

The intrinsic dissolution rate of the crystalline hydrochloride salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol is determined in aqueous media covering a range of pH 1.1-7.4 using the rotating disc method which maintains a constant surface area. 5 mg drug substance is compressed to form a disc at 356.1 N for 60 s. These discs are mounted to specially designed sample holders which fit into a Sotax dissolution tester. The dissolution media (37° C.) are stirred at 200 rpm. Samples are automatically withdrawn every second minute from the dissolution vessel and assayed via UV spectrophotometry. The intrinsic dissolution rate expressed in μg/cm²/min is calculated using the slope of the concentration versus time plot and from the linear portion of the slope of the dissolution curve, volume of dissolution medium (35 ml) and area (diameter: 2 mm) of the exposed disk. Intrinsic dissolution rates in aqueous media are shown in Table 3.

TABLE 3

| pH of aqueous medium | dissolution rate [μg/cm²/min] |
|---|---|
| water | 10 |
| 0.1N HCl | 230 |
| pH 2.2 (McIlvaine) | 250 |
| pH 3.0 (McIlvaine) | 60 |
| pH 4.0 (McIlvaine) | 10 |
| pH 4.5 (McIlvaine) | 10 |

From the above results, it can be concluded that for the crystalline hydrochloride salt form of (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol the intrinsic dissolution rates are fast to moderate up to pH 3.0 while at higher pH-values and also in water slow intrinsic dissolution rates are encountered.

### Crystalline Hydrobromide Salt of the Compound of Formula (I):

The following solid state characteristics and preparation of the crystalline hydrobromide salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, are typically relevant to the present invention.

Thus, the present invention relates to the crystalline hydrobromide salt form (particularly crystalline monohydrobromide salt form) of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, as may be characterized by one or more of the following characteristics.

Preparation of the Crystalline (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol Hydrobromide According to this Invention

Generally speaking the crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol is prepared by combining (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol (the free base of formula (I)) preferably dissolved or suspended in a suitable solvent and HBr (e.g. used as aqueous hydrobromic acid, or the like) at a suitable temperature (e.g. room temperature or elevated temperature) to provide the crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention, which is isolated and, optionally, purified, washed and/or dried.

Examples of such suitable solvents may include organic solvents such as ketones, e.g. acetone, methyl ethyl ketone or methyl isobutyl ketone, or ethers, e.g. diethyl ether, diisopropyl ether or tert.-butylmethylether, or the like, or mixtures thereof; with ketones and, especially, acetone being preferred.

In more detail, the present invention provides a method of making the crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol which comprises:

Contacting a solution of the compound of formula (I) (the free base) with HBr to form the hydrobromide salt of the compound of formula (I).

In certain embodiments, the solution of the compound of formula (I) comprises acetone. In other certain embodiments, the HBr is used as aqueous hydrobromic acid (48% in water).

In a further embodiment, the method further comprises crystallizing the crystalline hydrobromide salt form of the compound of formula (I) from the reaction solution. In certain embodiments, the crystals are precipitated, e.g. either spontaneously or being induced (e.g. by cooling, adding a non-solvent, concentration of the solvent(s) and/or, particularly, by (azeotropic) removal of water, if present; such as e.g. by codistillation of the acetone/water azeotrope).

In a yet further embodiment, the method further comprises isolating or collecting the crystals of the crystalline hydrobromide salt form of the compound of formula (I). In certain embodiments, the crystals are isolated by filtration.

In a still yet further embodiment, the method further comprises optionally washing and/or drying the isolated crystalline hydrobromide salt form of the compound of formula (I). In certain embodiments, the crystals are dried at a suitable temperature, e.g. at a temperature of about 30° C. In other certain embodiments, the crystals are dried under reduced pressure. The drying step may be conducted for a suitable period of time.

Solid State Characteristics of the Crystalline (3R,9S)-4-isopropyl-7,7-dimethyl-3-(4-(trifluoromethyl)phenyl)-2′,3,5′,6,6,7,8,9-octahydro-3H-spiro[furo[3,4-c]quinoline-1,4′-pyran]-9-ol Hydrobromide According to this Invention

Crystallinity and Polymorphism

This crystal form of the hydrobromide salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol is highly crystalline.

Bulk properties are favorable with high bulk density and good flowability. So far there are no indications for polymorphism for this hydrobromide salt form.

Under normal conditions the crystalline hydrobromide salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol according to the invention is present in an ansolvate and/or anhydrous (non-hydrate) form.

To study the hygroscopic behaviour of this material, sorption isotherms are registered on a DVS-1 water sorption monitor from Surface Measurement Systems. Adsorption and desorption isotherms are performed at 25° C. with 10% r.h. step intervals ranging from 10% r.h. up to 90% r.h.

It is found that the crystalline hydrobromide salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol according to the invention is not hygroscopic. Up to 90% r.h. an uptake of only about 0.9% of water is observed. No solid phase change is observed during moisture sorption/desorption.

Accordingly, in an embodiment, the present invention further relates to the crystalline hydrobromide salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol, characterised in that it is an anhydrous form.

In one embodiment, the thermoanalysis of the crystalline hydrobromide salt form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol according to the invention shows a T_{fus}=about 230° C. (DSC: 10 K·min⁻¹ heating rate) (ΔH_{fus}=about 110 J/g). Loss of drying is typically <0.5% release of water as shown by TG-IR coupling, indicating the anhydrous nature of this form.

In a further embodiment, the present invention further relates to the crystalline hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, having a fusion temperature of T_{fus}=about 230-232° C. (determined by DSC; heating rate: 10 K/min).

### Crystalline Free Base of the Compound of Formula (I):

The following solid state characteristics and preparation of the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, are typically relevant to the present invention.

Thus, the present invention relates to the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, as may be characterized by one or more of the following characteristics.

Preparation of Crystalline (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol According to this Invention

The present invention provides a method of making the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol which comprises:

Forming a solution of the compound of formula (I) (the free base) in a suitable organic solvent or mixture of organic solvents at a suitable temperature (e.g. room temperature or elevated temperature).

In certain embodiments, the solution of the compound of formula (I) comprises cyclohexane.

In a further embodiment, the method further comprises crystallizing the crystalline free base of the compound of formula (I) from the solution. In certain embodiments, the crystals are precipitated, e.g. either spontaneously or being induced (e.g. by cooling, adding a non-solvent or by concentration or evaporation of the solvent(s)).

In a yet further embodiment, the method further comprises isolating or collecting the crystals of the free base form of the compound of formula (I). In certain embodiments, the crystals are isolated by filtration.

In a still yet further embodiment, the method further comprises optionally washing and/or drying the isolated crystalline free base form of the compound of formula (I).

Solid State Characteristics of Crystalline (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol According to this Invention

Crystallinity and Polymorphism

This crystal form of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol is highly crystalline.

Under normal conditions the crystalline free base of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol according to the invention is present in an ansolvate and/or anhydrous (non-hydrate) form.

To study the hygroscopic behaviour of this material, sorption isotherms are registered on a DVS-1 water sorption monitor from Surface Measurement Systems. Adsorption and desorption isotherms are performed at 25° C. with 10% r.h. step intervals ranging from 10% r.h. up to 90% r.h.

It is found that the crystalline free base of (3R,9S)-4-isopropyl-7,7-dimethyl-3-(4-(trifluoromethyl)phenyl)-2′,3,5′,6,6′,7,8,9-octahydro-3H-spiro[furo[3,4-c]quinoline-1,4′-pyran]-9-ol according to the invention is not hygroscopic. Up to 90% r.h. an uptake of only about 0.2% of water is observed. No solid phase change is observed during moisture sorption/desorption.

Accordingly, in an embodiment, the present invention further relates to the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, characterised in that it is an anhydrous form.

The thermoanalysis of the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to the invention shows that this substance can exist in at least two crystalline forms. These two polymorphs are enantiotropically related and reversibly convert into each other approximately at 55° C. Under ambient conditions only the low temperature form is the thermodynamically stable form. Above the transformation temperature of about 55° C. the high temperature form is the thermodynamically stable form. In one embodiment, the high temperature form has T_{fus}=about 195±3° C. (DSC: 10 K·min⁻¹ heating rate) (ΔH_{fus}=about 67±5 J/g). Loss of drying is typically <0.5% release of water as shown by TG-IR coupling, indicating the anhydrous nature of the crystalline free base.

In a further embodiment, the present invention further relates to the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol having a fusion temperature of T_{fus}=about 190-195° C. (determined by DSC; heating rate: 10 K/min).

In a further embodiment, the present invention further relates to the crystalline free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol having a fusion temperature of T_{fus}=about 190-195° C. and which undergoes a reversible enantiotropic solid-state phase transition at about 55° C.

In a further embodiment, the present invention further relates to the crystalline low temperature form of free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol which undergoes a reversible solid-state phase transition at about 55° C. to the crystalline high temperature form thereof.

In a further embodiment, the present invention further relates to the crystalline high temperature form of free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol having a fusion temperature of T_{fus}=about 190-195° C. and which undergoes a reversible solid-state phase transition at about 55° C. to the crystalline low temperature form thereof.

In a further embodiment, the present invention further relates to a mixture (e.g. in any mixing ratio) of the crystalline low temperature form and crystalline high temperature form of free base of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

### Biological Assay

The biological properties of the compounds may be investigated as follows:

### CETP in Vitro Assay

CETP inhibitory activity of compounds of the present invention can be determined in a fluorometric assay purchased from Roar Biomedical, Inc. (New York, N.Y., USA). The compounds of the present invention inhibit CETP-dependent cholesterol ester transfer from HDL to LDL as described here. Recombinant human CETP was partially purified from medium conditioned by CETP expressing CHO cells. In a 384 well format 2.5 μl of compound solution in DMSO was combined with 2 μl of donor solution, 2 μl of acceptor solution and 0.8 μl of recombinant human CETP solution in a total volume of 100 μl with assay buffer and incubated for 3 hours at 37° C. The fluorescence intensity was measured at excitation wavelength of 485 nm and emission wavelength of 535 nm. IC₅₀ values are calculated from dose effect curves from compound concentrations between 1 nM and 30 μM.

The compound (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol for example has IC₅₀ value for inhibition of CETP of 18 nM.

### Indications

The compound of formula (I) and their physiologically tolerable salts according to the present invention have valuable pharmacological properties which make them commercially applicable. Thus, for example, these compounds can act as inhibitors of CETP and are expected to be commercially applicable in the therapy of diseases responsive to the inhibition of CETP, such as e.g. any of those diseases mentioned herein.

In view of their ability to inhibit enzyme cholesterol ester transfer protein (CETP), the compound of formula (I) according to the invention and the corresponding pharmaceutically acceptable salts thereof are theoretically suitable for the treatment and/or prevention of all those conditions or diseases which may be affected by the inhibition of the cholesterol ester transfer protein (CETP) activity. Therefore, compounds according to the invention, especially the crystalline HCl or HBr salt or free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention, are particularly suitable for the treatment and/or prevention of cardiovascular and/or cardiometabolic and related disorders, in particular atherosclerosis, peripheral vascular disease, dyslipidemia (including e.g. mixed dyslipidemia), hyperbeta-lipoproteinemia, hypoalpha-lipoproteinemia, hypercholesterolemia, hypertriglyceridemia, hyperlipidemia, hypolipoproteinemia, hyperlipoproteinemia, hypo-HDL cholesterolemia, hyper-LDL cholesterolemia, familial hypercholesterolemia, angina, ischemia, cardiac ischemia, stroke, myocardial infarction, reperfusion injury, angioplastic restenosis, hypertension, endothelial dysfunction, vascular complications of diabetes, prevention of diabetes, insulin resistance, obesity, metabolic syndrome, diabetes (especially type 2 diabetes mellitus) or endotoxemia, or arteriosclerosis, coronary heart disease, coronary artery disease, coronary vascular disease or congestive heart failure.

### Application Forms and Dosages

The crystalline forms according to this invention can be used as medicaments, e.g. in the form of pharmaceutical compositions for enteral, parenteral or topical administration. They may be administered in any of the generally accepted modes of administration available in the art, e.g., perorally, e.g. in the form of tablets, coated tablets, dragees, hard and soft gelatine capsules, solutions, emulsions or suspensions, rectally, e.g. in the form of suppositories, parenterally (including intravenously), e.g. in the form of injection solutions or infusion solutions, or topically, e.g. in the form of ointments, creams or oils. Among the possible modes of administration, oral and intravenous delivery are preferred; with oral administration being particularly preferred.

The pharmaceutical compositions according to this invention contain at least one of the compounds of the invention (=active compound), e.g. in a total amount of from 0.1 to 99.9 wt %, 5 to 95 wt %, or 20 to 80 wt %, optionally together with pharmaceutically acceptable excipients.

The person skilled in the art is familiar with pharmaceutically acceptable excipients, such as e.g. diluents, carriers, binders, disintegrants, surfactants, lubricants, vehicles, auxiliaries, adjuvants and/or further additives which are known to be suitable for preparing pharmaceutical compositions, on account of his/her expert knowledge.

As pharmaceutically acceptable excipients, usually any excipients known to be appropriate for pharmaceutical compositions come into consideration. Examples thereof include, but are not limited to, diluents, fillers, binders, disintegrants, lubricants, glidants, solvents, dispersants, emulsifiers, solubilizers, gel formers, ointment bases, antioxidants, preservatives, stabilizers, carriers, thickeners, complexing agents, buffers, pH regulators (e.g. to obtain neutral, alkaline or acidic formulations), permeation promoters, polymers, coating agents, propellants, tonicity adjusting agents, surfactants, colorants, flavorings, sweeteners and dyes.

In general, suitable carrier materials are not only inorganic carrier materials, but also organic carrier materials. Thus, e.g., lactose, starches (e.g. corn starch) or derivatives thereof, talc, silica, polyvinylpyrrolidones, stearic acid or its salts can be used as carrier materials for tablets, coated tablets, dragees and hard gelatine capsules. Suitable carrier materials for soft gelatine capsules are, e.g., vegetable oils, waxes, fats and semi-solid and liquid polyols. Suitable carrier materials for the production of solutions and syrups are, e.g., water, polyols, sucrose, invert sugar and the like. Suitable carrier materials for injection or infusion solutions are, e.g., water, alcohols, polyols, glycerol and vegetable oils. Suitable carrier materials for suppositories are, e.g., natural or hardened oils, waxes, fats and semi-liquid or liquid polyols or polyethylene glycols.

Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

In particular, excipients, carriers and/or diluents of a type appropriate to the desired pharmaceutical composition, formulation or preparation and the desired mode of administration are used.

The pharmaceutical compositions according to this invention can be prepared by processes which are known per se and familiar to the person skilled in the art, e.g. by incorporating the described crystalline forms according to this invention (optionally combined with other active substances) optionally together with one or more conventional carriers (e.g. solid or liquid carriers) and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, into conventional galenic preparations such as plain or coated tablets, capsules, powders, suspensions or suppositories.

The dosage of the compounds of the invention (=active compounds) can vary within wide limits depending on the compound which is to be administered, the nature and gravity of the disease to be treated or prevented, the age and the individual condition of the patient and the mode and frequency of administration, and will, of course, be fitted to the individual requirements in each particular case. Usually, a dosage of the compounds of the invention (=active compounds) in the order of magnitude customary for CETP inhibitors comes into consideration. Expediently, the dosage may be from 0.1 ng/ml to 10 mg/ml, preferably 1 ng/ml to 10 mg/ml, by intravenous route, and 0.1 to 2000 mg, preferably 1 to 100 mg, by oral route, in each case administered 1 to 4 times a day. Depending on the dosage it may be convenient to administer the daily dosage in several dosage units.

### Combinations

Besides their use in monotherapy, the compounds according to the invention, especially the crystalline HCl or HBr salt or free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol according to this invention, may also be used in conjunction with other active substances, particularly for the treatment and/or prevention of the diseases, disorders and conditions mentioned above.

Other active substances which are suitable for such a combination include for example those which potentiate the therapeutic effect of a cholesterol ester transfer protein (CETP) inhibitor according to the invention with respect to one of the indications mentioned and/or which allow the dosage of a cholesterol ester transfer protein (CETP) inhibitor according to the invention to be reduced.

Therapeutic agents which are suitable for such a combination include particularly one or more lipid modulating agents. Lipid modulating agents comprise HMG CoA reductase inhibitors (e.g. simvastatin, atorvastatin), fibrates (e.g. bezafibrate, fenofibrate), nicotinic acid and the derivatives thereof, PPAR (α, γ or α/γ) agonists or modulators, ACAT inhibitors (e.g. avasimibe), MTP inhibitors, squalene cyclase and squalene synthase inhibitors, LXR agonists or modulators, bile acid-binding substances such (e.g. cholestyramine, colesevelam), cholesterol absorption inhibitors (e.g. ezetimibe), niacin, PCSK9 inhibitors, bile acid reuptake inhibitors and lipase inhibitors.

Other therapeutic agents which are suitable for such a combination include one or more antidiabetic agents as for example metformin, alpha-glucosidase inhibitors (e.g. acarbose, voglibose), PPAR (α, γ or α/γ) agonists or modulators, DPP-IV inhibitors (e.g. Sitagliptin, Vildagliptin, Saxagliptin, Alogliptin, Linagliptin), SGLT 2 inhibitors (e.g. dapagliflozin, sergliflozin), GLP-1 or GLP-1 analogues (e.g. exenatide, liraglutide), insulin or insulin analogues, sulphonylureas (e.g. glibenclamide, tolbutamide, glimepiride), thiazolidinediones (e.g. rosiglitazone, pioglitazone), nateglinide, repaglinide, II-β-HSD inhibitors, glucose-6-phosphatase inhibitors, fructose-1,6-bisphosphatase inhibitors, glycogen phosphorylase inhibitors, glucagon receptor antagonists, inhibitors of phosphoenol pyruvate carboxykinase, glycogen synthase kinase or pyruvate dehydrokinase and glucokinase activators.

Also suitable for such a combination are one or more antiobesity agents including for example sibutramine, tetrahydrolipostatin, leptin, leptin mimetics, antagonists of the cannabinoid) receptor, MCH-1 receptor antagonists, MC4 receptor agonists, NPY5 or NPY2 antagonists or β3-agonists such as SB-418790 or AD-9677 and agonists of the 5HT2c receptor.

Moreover, combinations with drugs for influencing high blood pressure or chronic heart failure such as e.g. A-II antagonists or ACE inhibitors, ECE inhibitors, diuretics, β-blockers, Ca-antagonists, centrally acting antihypertensives, antagonists of the alpha-2-adrenergic receptor, inhibitors of neutral endopeptidase, thrombocyte aggregation inhibitors and others or combinations thereof are suitable. Examples of angiotensin II receptor antagonists are candesartan cilexetil, potassium losartan, eprosartan mesylate, valsartan, telmisartan, irbesartan, EXP-3174, L-158809, EXP-3312, olmesartan, medoxomil, tasosartan, KT-3-671, GA-0113, RU-64276, EMD-90423, BR-9701, etc. Angiotensin II receptor antagonists are preferably used for the treatment or prevention of high blood pressure and complications of diabetes, often combined with a diuretic such as hydrochlorothiazide.

The therapeutic agents mentioned herein above as combination partners of the compounds according to this invention are meant to include pharmaceutically acceptable derivatives thereof, such as e.g. their pharmaceutically acceptable salts. The person skilled in the art is aware on the base of his/her expert knowledge of the kind, total daily dosage(s) and administration form(s) of the additional therapeutic agent(s) coadministered. Said total daily dosage(s) can vary within a wide range. Usually, the dosage for the combination partners mentioned above is ⅕ of the lowest dose normally recommended up to 1/1 of the normally recommended dose.

In practicing the present invention, the compounds according to this invention may be administered in combination therapy separately, sequentially, simultaneously, concurrently or chronologically staggered with one or more further active substances, such as e.g. any of the therapeutic agents mentioned herein above as a combination partner.

In this context, the present invention further relates to a combination comprising a first active ingredient, which is at least one crystalline form according to this invention, and a second active ingredient, which is at least one of the active substances described above as a combination partner, for separate, sequential, simultaneous, concurrent or chronologically staggered use in therapy, particularly for treatment and/or prevention of cardiovascular or related disorders, such as e.g. any of those mentioned herein.

Further, this invention relates to the use of a crystalline form according to this invention combined with at least one of the active substances described above as a combination partner, for preparing a pharmaceutical composition which is suitable for the treatment or prevention of diseases or conditions which may be affected by the inhibition of the cholesterol ester transfer protein (CETP) activity, particularly cardiometabolic and/or cardiovascular disorders, more particularly one of the diseases, disorders or conditions listed above.

Further, this invention relates to a pharmaceutical composition which comprises a crystalline form according to the invention and at least one of the active substances described above as combination partners, optionally together with one or more inert carriers and/or diluents.

The term “combination” according to this invention may be present as a fixed combination, a non-fixed combination, a free combination or a kit-of-parts.

A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously, concurrently or chronologically staggered.

The first and second active ingredient of a kit-of-parts according to this invention may be provided as separate formulations (i.e. independently of one another), which are subsequently brought together for simultaneous, concurrent, sequential, separate or chronologically staggered use in combination therapy; or packaged and presented together as separate components of a combination pack for simultaneous, concurrent, sequential, separate or chronologically staggered use in combination therapy.

The type of pharmaceutical formulation of the first and second active ingredient of a kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration.

The amounts of the first and second active ingredients of the combinations, compositions or kits according to this invention may together comprise a therapeutically effective amount, particularly for the treatment and/or prevention of the diseases, disorders and conditions mentioned above.

### Synthesis

The compounds according to the invention may be obtained using methods of synthesis known in principle. Preferably the compounds are obtained by the following methods according to the invention which are described in more detail hereinafter.

As already mentioned hereinbefore, the compound (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol is specifically disclosed in unpublished patent application PCT/EP2011052376 (U.S. Ser. No. 13/029,697, U.S. Ser. No. 13/029,690) as well as a process for its preparation. For details on a process to manufacture this compound, reference is thus made to PCT/EP2011052376 (U.S. Ser. No. 13/029,697, U.S. Ser. No. 13/029,690) as well as to the process disclosed in the present application (e.g. process a) as outlined in scheme 1 below and/or described by way of example in the following examples).

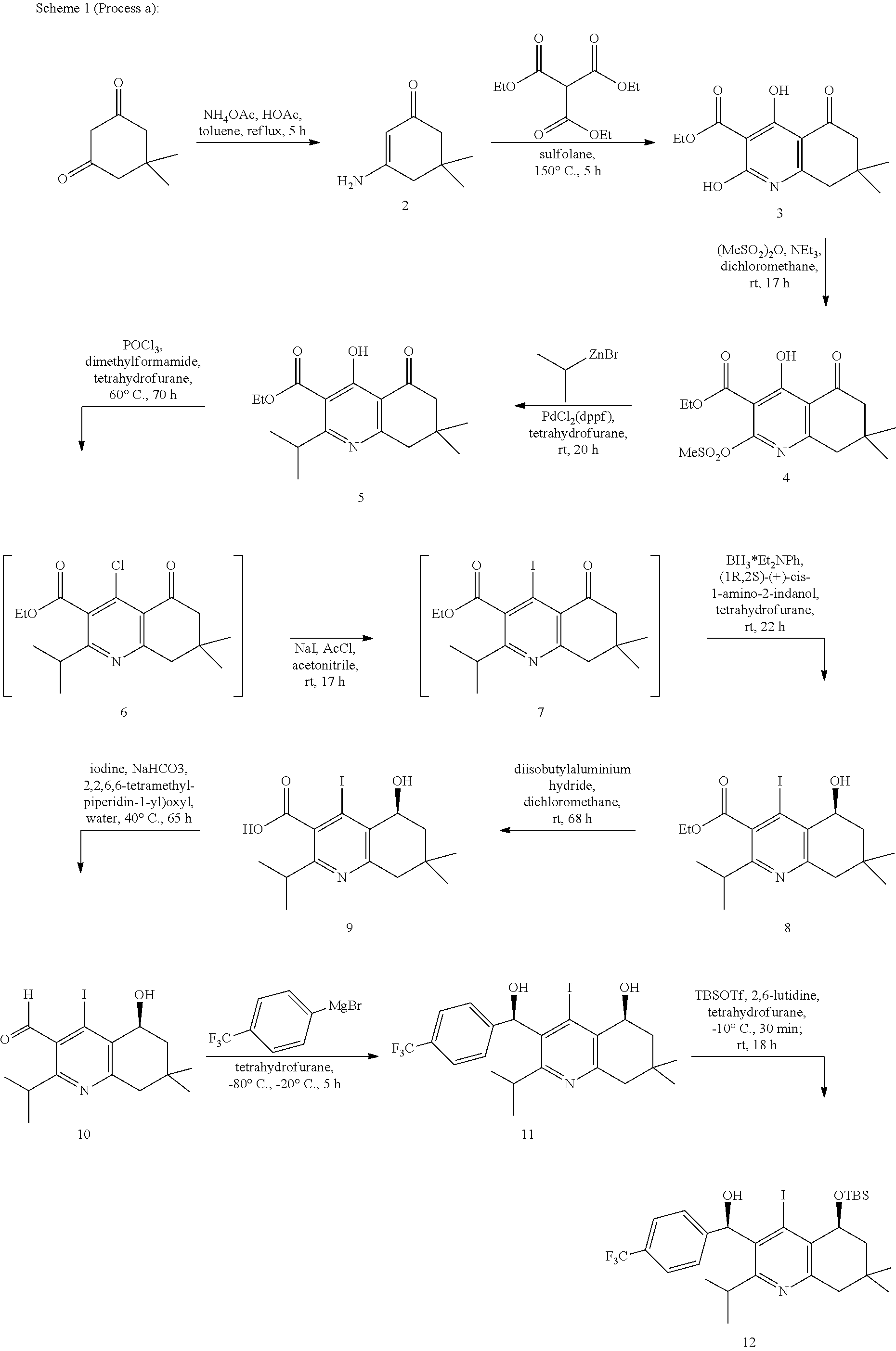

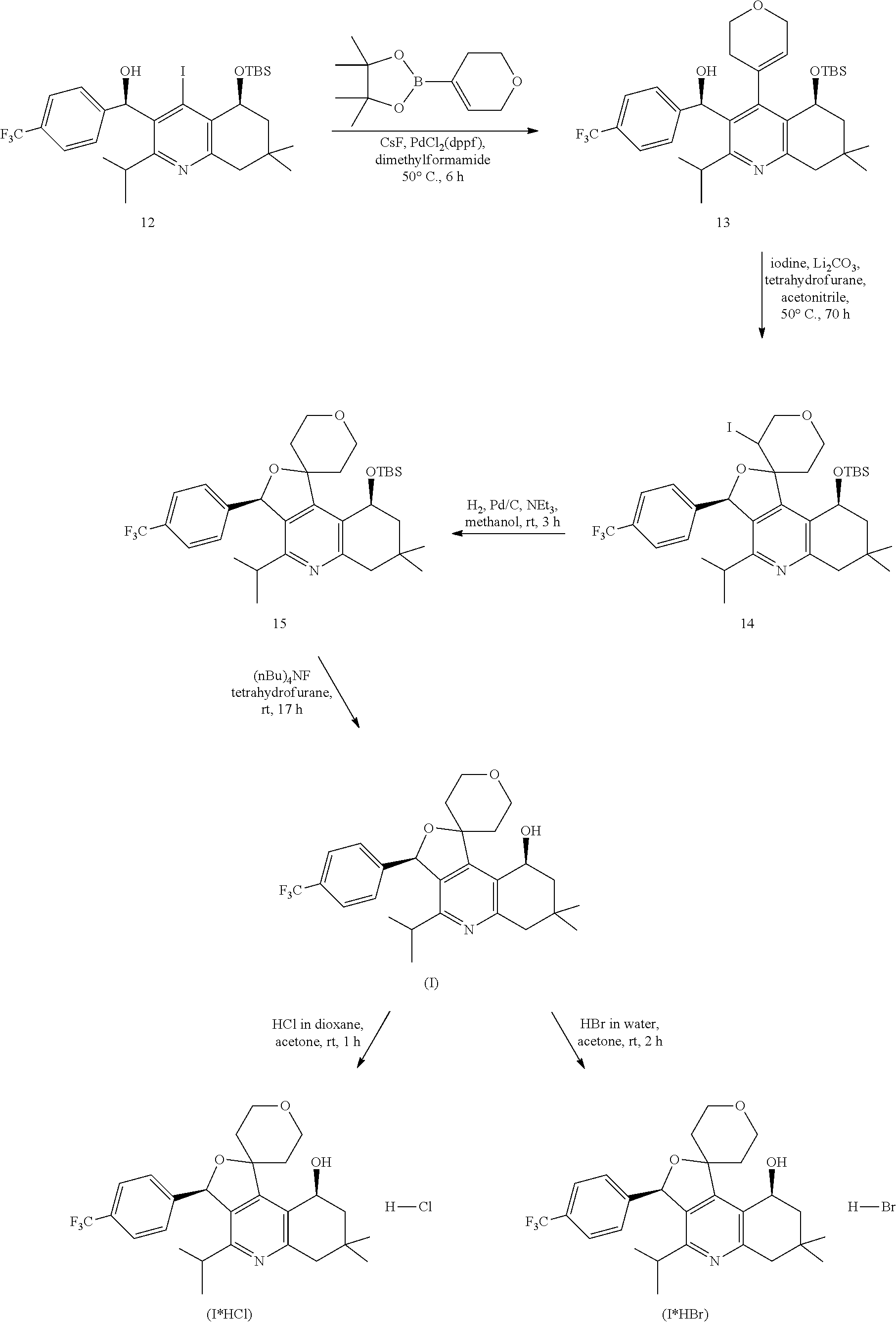

Besides the synthesis strategies presented a host of additional approaches can be envisaged. Therefore, the preceding strategies are in no way meant to restrict the possible synthetic pathways to access the compounds of the invention but are only supposed to show a route by way of example.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesis” by T. Greene and P. Wuts (John Wiley & Sons, Inc. 1999, 3rd Ed.) or in “Protecting Groups (Thieme Foundations Organic Chemistry Series N Group” by P. Kocienski (Thieme Medical Publishers, 2000).

The present invention also relates to intermediates (including their tautomers, stereoisomers (e.g. enantiomers, diastereomers), mixtures and salts thereof), methods, steps and processes which are disclosed herein and which are useful in synthesizing final compounds according to this invention. Thus, the present invention also relates to synthesis steps or processes disclosed herein for preparing compounds or intermediates according to this invention, which synthesis steps or processes may be performed as described herein. Said synthesis steps may comprise any or all of the respective reaction features as disclosed herein. Said processes may comprise one or more steps of converting and/or reacting the mentioned intermediates with the appropriate reaction partners, suitably under conditions as disclosed herein.

In general, all isomeric forms (especially all regio- and stereoisomeric forms, e.g. all chiral, enantiomeric, diastereomeric, racemic forms, tautomeric and all geometric isomeric forms) of a compound mentioned herein are intended within this invention, unless the specific isomer is specifically disclosed.

Compounds obtained according to the synthesis steps of this invention (e.g. intermediates) may be resolved into their enantiomers and/or diastereomers, e.g. as mentioned herein. Thus, for example, diastereomeric mixtures may be resolved into their respective diastereomers, and racemic compounds may be separated into their enantiomers.

Substantially pure stereoisomers (e.g. diastereomers and/or enantiomers) can be obtained according to synthetic principles customary to the skilled person, e.g. by separation of corresponding mixtures, by using stereochemically pure starting materials and/or by stereoselective synthesis.

It is known in the art how to prepare optically active forms, such as by resolution of racemic forms or by synthesis, e.g. from optically active starting materials and/or by using chiral reagents.

Enantiomerically pure compounds can be prepared via asymmetric synthesis, for example by preparation and separation of appropriate diastereoisomeric compounds/intermediates which can be separated by known methods (e.g. by chromatographic separation or (fractional) crystallization from a suitable solvent), and/or by using chiral reaction components (e.g. chiral reagents, chiral catalysts, chiral ligands, chiral synthons, chiral building blocks, or the like).

Further, it is known to the person skilled in the art how to prepare enantiomerically pure compounds from the corresponding racemic mixtures, such as e.g. by chromatographic separation of the corresponding racemic compounds on chiral separating columns; or by resolution of racemic compounds using an appropriate resolving agent; e.g. by means of diastereomeric salt formation of the racemic compounds with optically active acids or bases, subsequent resolution of the salts and release of the desired compound from the salt; or by derivatization of the corresponding racemic compounds with chiral auxiliary reagents, subsequent diastereomer separation and removal of the chiral auxiliary group; by kinetic resolution of a racemate (e.g. by enzymatic resolution); by enantioselective (preferential) crystallization (or crystallization by entrainment) from a conglomerate of enantiomorphous crystals under suitable conditions; or by (fractional) crystallization from a suitable solvent in the presence of a chiral auxiliary.

For example, the diastereomeric or cis/trans mixtures may be resolved by chromatography into the respective diastereomers or cis and trans isomers thereof. The compounds which occur as racemates may be separated by methods known per se (cf. Allinger N. L. and Eliel E. L. in “Topics in Stereochemistry”, Vol. 6, Wiley Interscience, 1971) into their optical antipodes and compounds with at least 2 asymmetric carbon atoms may be resolved into their diastereomers on the basis of their physical-chemical differences using methods known per se, e.g. by chromatography and/or fractional crystallization, and, if these compounds are obtained in racemic form, they may subsequently be resolved into the enantiomers as mentioned above.

The racemates may be for example resolved by column chromatography on chiral phases or by crystallization from an optically active solvent or by reacting with an optically active substance which forms salts or derivatives such as esters or amides with the racemic compound. Salts may be formed with enantiomerically pure acids for basic compounds and with enantiomerically pure bases for acidic compounds. Diastereomeric derivatives are formed with enantiomerically pure auxiliary compounds, e.g. acids, their activated derivatives, or alcohols. Separation of the diastereomeric mixture of salts or derivatives thus obtained may be achieved by taking advantage of their different physico-chemical properties, e.g. differences in solubility; the free antipodes may be released from the pure diastereomeric salts or derivatives by the action of suitable agents. Optically active acids in common use for such a purpose are e.g. the D- and L-forms of tartaric acid, dibenzoyltartaric acid, ditoloyltartaric acid, malic acid, mandelic acid, camphorsulfonic acid, glutamic acid, aspartic acid, or quinic acid. Optically active alcohols applicable as auxiliary residues may be, for example, (+) or (−)-menthol and optically active acyl groups in amides may be, for example, (+)—or (−)-menthyloxycarbonyl.

Moreover, the present invention further includes the products obtainable from the processes or synthesis steps disclosed herein.

The compounds of this invention are obtainable using the methods described in the examples that follow, which may also be combined for this purpose with methods known to the skilled person from his/her expert knowledge.

Any or all of the compounds or crystalline forms according to the present invention which are obtained as described in the following examples (particularly as final compounds) are a particularly interesting subject within the present invention.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### Examples

### 3-Amino-5,5-dimethyl-2-cyclohexen-1-one

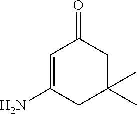

3-Amino-5,5-dimethyl-2-cyclohexen-1-one can be synthesized as known from literature or can be obtained by the following synthesis:

A mixture of 200 g dimedone, 143 g ammonium acetate, and 5 ml acetic acid in 2 L toluene is refluxed for 5 h at a Dean-Stark trap. Then the solution is cooled to room temperature and stirred for 16 h. The suspension is filtered and the filter cake is dried at 65° C.

Yield: 171 g (86% of theory)

Mass spectrometry (ESI⁺): m/z=140 [M+H]⁺

R_{f}-value: 0.31 (silica gel, dichloromethane/ethanol 9:1)

### Ethyl 2,4-dihydroxy-7,7-dimethyl-5-oxo-5,6,7,8-tetrahydroquinoline-3-carboxylate

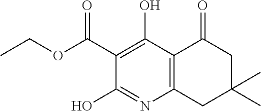 [SMILES: CCOC(=O)c1c(O)nc2c(c1O)C(=O)CC(C)(C)C2]

150 g 3-amino-5,5-dimethyl-2-cyclohexen-1-one and 250 ml 2-ethoxycarbonyl-malonic acid diethyl ester in 100 ml sulfolane are heated to 150° C. Volatiles are continuously distilled of at 100 mbar. After 5 h the reaction mixture is cooled to 100° C. and treated with 70 ml dioxane. Then the reaction mixture is cooled to 50° C. and treated with 500 ml methyl tert-butyl ether. The mixture is stirred at room temperature for 15 h and then the precipitate is collected by filtration and dried at 70° C.

Yield: 163 g (54% of theory)

Mass spectrometry (ESI⁺): m/z=280 [M+H]⁺

R_{f}-value: 0.24 (silica gel, dichloromethane/methanol 20:1)

### Ethyl 4-hydroxy-2-methanesulfonyloxy-7,7-dimethyl-5-oxo-5,6,7,8-tetrahydroquinoline-3-carboxylate

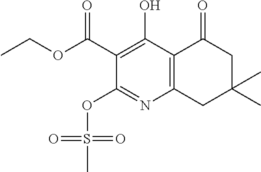 [SMILES: CCOC(=O)c1c(OS(C)(=O)=O)nc2c(c1O)C(=O)CC(C)(C)C2]

To a solution of 90 g ethyl 2,4-dihydroxy-7,7-dimethyl-5-oxo-5,6,7,8-tetrahydroquinoline-3-carboxylate and 55 ml triethylamine in 400 ml dichloromethane is added portion wise 73 g methanesulfonic anhydride at room temperature. After stirring for 17 h, 20 g methanesulfonic anhydride and 15 ml triethylamine are added. After 3 h the reaction mixture is diluted with 200 ml tetrahydrofuran and then 100 ml aqueous sodium hydrogen carbonate solution (1 M) are slowly added. The mixture is heated to 50° C. for 2 h, then cooled to room temperature, treated with 20 g sodium hydrogen carbonate, and then heated to 50° C. for 1 h. After 65 h at room temperature, The reaction mixture is concentrated in vacuo and then treated with 500 ml ethyl acetate. The suspension is filtered and the filtrate is concentrated in vacuo. The residue is chromatographed on silica gel (cyclohexane/ethyl acetate 8:1).

Yield: 54 g (47% of theory)

Mass spectrometry (ESI⁺): m/z=358 [M+H]⁺

R_{f}-value: 0.86 (silica gel, dichloromethane/methanol 20:1)

R_{f}-value: 0.53 (silica gel, cyclohexane/ethyl acetate 1:1)

### Ethyl 4-hydroxy-2-isopropyl-7,7-dimethyl-5-oxo-5,6,7,8-tetrahydroquinoline-3-carboxylate

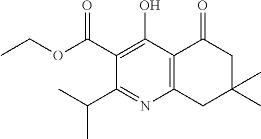 [SMILES: CCOC(=O)c1c(C(C)C)nc2c(c1O)C(=O)CC(C)(C)C2]

To a solution of 50 g ethyl 4-hydroxy-2-methanesulfonyloxy-7,7-dimethyl-5-oxo-5,6,7,8-tetrahydroquinoline-3-carboxylate in 100 ml tetrahydrofurane are added 2 g 1,1′-bis-(diphenylphosphino)-ferrocene-dichloro-palladium-(II). At room temperature 500 ml of a solution of 2-propylzinc bromide in tetrahydrofurane (0.5 M) are added during 30 min. After 20 h at room temperature 20 ml water and 30 ml acetic acid are slowly added and the mixture is stirred for 40 min. 300 ml ethyl acetate are added and 300 ml solvent are removed by evaporation. The residue is firstly chromatographed on silica gel (cyclohexane/ethyl acetate/acetic acid 50:50:1). The obtained crude product is then dissolved in 300 ml ethanol at reflux and treated with 300 ml water. After cooling and seeding with product, the suspension is stirred for 20 h. The precipitate is collected by filtration and dried at 50° C.

Yield: 36 g (84% of theory)

Mass spectrometry (ESI⁺): m/z=306 [M+H]⁺

R_{f}-value: 0.78 (silica gel, cyclohexane/ethyl acetate 1:1)

### Ethyl 4-chloro-2-isopropyl-7,7-dimethyl-5-oxo-5,6,7,8-tetrahydroquinoline-3-carboxylate

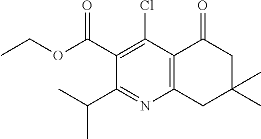 [SMILES: CCOC(=O)c1c(C(C)C)nc2c(c1Cl)C(=O)CC(C)(C)C2]

To a solution of 36 g ethyl 4-hydroxy-2-isopropyl-7,7-dimethyl-5-oxo-5,6,7,8-tetrahydroquinoline-3-carboxylate and 20 ml dimethylformamide in 400 ml tetrahydrofurane are added 25 ml phosphoryl chloride and the mixture is heated to 60° C. After 68 h 10 ml phosphoryl chloride are added and the mixture is heated to 70° C. After 2 h the reaction mixture is cooled to room temperature and then poured onto 300 ml ice water. The aqueous layer is extracted with 200 ml ethyl acetate and 100 ml cyclohexane. The organic layer is separated and concentrated in vacuo to yield 66 g of impure crude product, which is used directly in the next chemical step without further purification.

In an alternative version, tetrahydrofurane can be replaced by an excess of phosphoryl chloride to obtain the compound in higher purity.

### Ethyl 4-iodo-2-isopropyl-7,7-dimethyl-5-oxo-5,6,7,8-tetrahydroquinoline-3-carboxylate

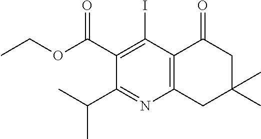 [SMILES: CCOC(=O)c1c(C(C)C)nc2c(c1I)C(=O)CC(C)(C)C2]

To a solution of 130 g of crude ethyl 4-chloro-2-isopropyl-7,7-dimethyl-5-oxo-5,6,7,8-tetrahydroquinoline-3-carboxylate (potency approx. 35%) in 400 ml acetonitrile are added 80 g sodium iodide and 2 ml acetyl chloride and the reaction mixture is stirred at room temperature for 17 h. After stirring for 2 h at 50° C., the reaction mixture is cooled to 0° C. and 100 ml aqueous sodium hydrogen carbonate solution (1 M) are slowly added. Then the organic solvent is removed by evaporation in vacuo at 40° C. 200 ml water is added and the mixture is extracted five times with cyclohexane (100 ml each). The combined organic layers are concentrated in vacuo to yield 106 g of impure crude product, which is used directly in the next chemical step without further purification.

### (S)-Ethyl 5-hydroxy-4-iodo-2-isopropyl-7,7-dimethyl-5,6,7,8-tetrahydroquinoline-3-carboxylate

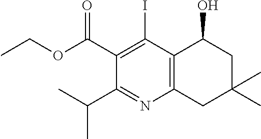 [SMILES: CCOC(=O)c1c(C(C)C)nc2c(c1I)[C@@H](O)CC(C)(C)C2]

1 g (1R,2S)-(+)-cis-1-amino-2-indanol is dissolved in 300 ml tetrahydrofurane and to this solution are added 20 ml of a borane-diethylaniline-complex. After 20 min the solution is cooled to 0° C. and 106 g of crude ethyl 4-iodo-2-isopropyl-7,7-dimethyl-5-oxo-5,6,7,8-tetrahydroquinoline-3-carboxylate (potency approx. 40%) in 50 ml tetrahydrofurane are added drop wise within 10 min. After 22 h at room temperature 50 ml methanol are added dropwise and the mixture is stirred for additional 10 minutes. The solvents are evaporated in vacuo and the residue is firstly chromatographed on silica gel (cyclohexane/ethyl acetate 30:1). The resulting crude mixture is then again chromatographed on silica gel (cyclohexane/ethyl acetate 30:1).

Yield: 33 g (32% of theory over 3 steps)

Mass spectrometry (ESI⁺): m/z=418 [M+H]⁺

R_{f}-value: 0.27 (silica gel, cyclohexane/ethyl acetate 5:1)

### (S)-3-(hydroxymethyl)-4-iodo-2-isopropyl-7,7-dimethyl-5,6,7,8-tetrahydroquinolin-5-01

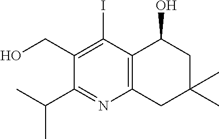 [SMILES: CC(C)c1nc2c(c(I)c1CO)[C@@H](O)CC(C)(C)C2]

To a solution of 80 g (S)-ethyl 5-hydroxy-4-iodo-2-isopropyl-7,7-dimethyl-5,6,7,8-tetrahydroquinoline-3-carboxylate in 400 ml dichloromethane are added 400 ml of a 1M diisobutylaluminium hydride solution in dichloromethane at 0° C. within 1 h. After 2 h at room temperature 100 ml of a 1M diisobutylaluminium hydride solution in dichloromethane are added. After 19 h at room temperature 100 ml of a 1M diisobutylaluminium hydride solution in dichloromethane are added. After 24 h at room temperature 100 ml of a 1M diisobutylaluminium hydride solution in dichloromethane are added. After 23 h at room temperature 100 ml of an aqueous HCl solution (2 M) is added at 0° C. After 1 h 50 ml of an aqueous sodium hydrogen carbonate solution (1 M) are slowly added. After 2 h the mixture is concentrated in vacuo and the residue in suspended in ethyl acetate. The suspension is filtered through silica gel (eluent: ethyl acetate). The solvent is evaporated and the residue is re-crystallized from 300 ml toluene. 55 g of the crude product are dissolved in 400 ml diethyl ether at 36° C. 80 ml pentane are added and the solution is cooled to 0° C. The resulting suspension is stirred for 2 h and then filtered. The filter cake is dried in vacuo at 40° C.

Yield: 38 g (53% of theory)

Mass spectrometry (ESI⁺): m/z=376 [M+H]⁺

R_{f}-value: 0.08 (silica gel, cyclohexane/ethyl acetate 5:1)

### (S)-5-hydroxy-4-iodo-2-isopropyl-7,7-dimethyl-5,6,7,8-tetrahydroquinoline-3-carbaldehyde

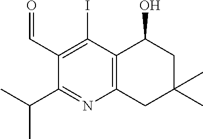 [SMILES: CC(C)c1nc2c(c(I)c1C=O)[C@@H](O)CC(C)(C)C2]

To a solution of 103 g (S)-3-(hydroxymethyl)-4-iodo-2-isopropyl-7,7-dimethyl-5,6,7,8-tetrahydroquinolin-5-ol in 1 L toluene are sub sequentially added 1.5 L aqueous sodium hydrogen carbonate solution (0.5 M), 139 g iodine, and 2 g (2,2,6,6-tetramethylpiperidin-1-yl)oxyl (TEMPO). After 65 h at 40° C. the reaction mixture is cooled to 0° C. and 1 L methyl tert-butyl ether and 250 ml aqueous sodium thiosulfate solution (2 M) are added. The organic layer is separated. To the aqueous layer 300 g sodium chloride are added and the mixture is extracted three times with 200 ml ethyl acetate each. The combined organic layers are evaporated in vacuo and the residue is chromatographed on silica gel (cyclohexane/ethyl acetate 1:1).

Yield: 89 g (87% of theory)

Mass spectrometry (ESI⁺): m/z=374 [M+H]⁺

R_{f}-value: 0.34 (silica gel, cyclohexane/ethyl acetate 5:1)

### (S)-3-((R)-hydroxy(4-(trifluoromethyl)phenyl)methyl)-4-iodo-2-isopropyl-7,7-dimethyl-5,6,7,8-tetrahydroquinolin-5-ol

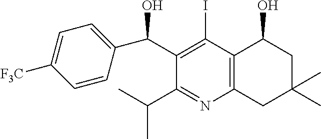 [SMILES: CC(C)c1nc2c(c(I)c1[C@H](O)c1ccc(C(F)(F)F)cc1)[C@@H](O)CC(C)(C)C2]

To a stirred suspension of 16.6 g magnesium and 20 mg iodine in 2.8 L tetrahydrofurane are added drop wise 95.6 ml 4-bromobenzotrifluoride over 30 min at 35° C. After 2 h the mixture is cooled to −80° C. and a solution of 85 g (S)-5-hydroxy-4-iodo-2-isopropyl-7,7-dimethyl-5,6,7,8-tetrahydroquinoline-3-carbaldehyde in 200 ml tetrahydrofurane is added over 30 min. The temperature is slowly raised to −20° C. over 4 h, kept at −20° C. for 40 min, and then cooled to −80° C. 50 ml MeOH are slowly added and the reaction mixture is stirred at room temperature for 64 h. The solvent is evaporated in vacuo and the residue is treated with ethyl acetate. After filtration the filtrate is evaporated in vacuo and the residue is firstly chromatographed on silica gel (ethyl acetate). The resulting mixture of isomers is then again chromatographed on silica gel (cyclohexane/ethyl acetate 10:1).

Yield: 75.0 g (63% of theory)

Mass spectrometry (ESI⁺): m/z=520 [M+H]⁺

R_{f}-value: 0.24 (silica gel, cyclohexane/ethyl acetate 5:1)

### (R)-((S)-5-(tert-butyldimethylsilyloxy)-4-iodo-2-isopropyl-7,7-dimethyl-5,6,7,8-tetrahydroquinolin-3-yl)(4-(trifluoromethyl)phenyl)methanol

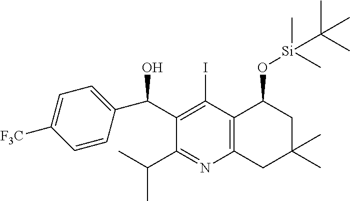 [SMILES: CC(C)c1nc2c(c(I)c1[C@H](O)c1ccc(C(F)(F)F)cc1)[C@@H](O[Si](C)(C)C(C)(C)C)CC(C)(C)C2]

To a solution of 28 g (S)-3-((R)-hydroxy(4-(trifluoromethyl)phenyl)methyl)-4-iodo-2-isopropyl-7,7-dimethyl-5,6,7,8-tetrahydroquinolin-5-ol in 280 ml tetrahydrofurane are added 6.6 ml 2,6-lutidine. The solution is cooled to −10° C. and 13 ml tert-butyldimethyl trifluoromethanesulfonate are added portion wise over 1 h. The reaction mixture is stirred for 30 min at −10° C. and then 18 h at room temperature. The solvent is evaporated in vacuo and the residue is chromatographed on silica gel (cyclohexane/ethyl acetate 50:1).

Yield: 31.7 g (93% of theory)

Mass spectrometry (ESI⁺): m/z=634 [M+H]⁺

R_{f}-value: 0.59 (silica gel, cyclohexane/ethyl acetate 5:1)

### (R)(S)-5-(tert-butyldimethylsilyloxy)-4-(3,6-dihydro-2H-pyran-4-yl)-2-isopropyl-7,7-dimethyl-5,6,7,8-tetrahydroquinolin-3-yl)(4-(trifluoromethyl)phenyl)methanol

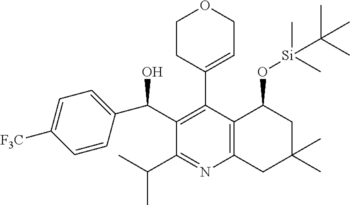 [SMILES: CC(C)c1nc2c(c(C3=CCOCC3)c1[C@H](O)c1ccc(C(F)(F)F)cc1)[C@@H](O[Si](C)(C)C(C)(C)C)CC(C)(C)C2]

To a solution of 40 g (R)-((S)-5-(tert-butyldimethylsilyloxy)-4-iodo-2-isopropyl-7,7-dimethyl-5,6,7,8-tetrahydroquinolin-3-yl)(4-(trifluoromethyl)phenyl)methanol in 500 ml tetrahydrofurane are added 24 g cesium fluoride. The suspension is evaporated to dryness. The residue is dissolved in 400 ml dimethylformamide and 24 g 2-(3,6-dihydro-2H-pyran-4-yl)-4,4,5,5-tetramethyl-1,3,2-dioxaborolane are added. After the addition of 2.4 g of 1,1′-bis-(diphenylphosphino)-ferrocene-dichloro-palladium-(II) the mixture is heated to 50° C. for 5.5 hours. The reaction mixture is cooled to room temperature and added to 2000 ml water. After 20 min of stirring the suspension is filtered and the filter cake dried in vacuo at 30° C. The crude product is chromatographed on silica gel (cyclohexane/ethylacetate 5:1).

Yield: 30.1 g (81% of theory)

Mass spectrometry (ESI⁺): m/z=590 [M+H]⁺

R_{f}-value: 0.38 (silica gel, cyclohexane/ethyl acetate 5:1)

### (3R,9S)-9-(tert-butyldimethylsilyloxy)-3′-iodo-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]

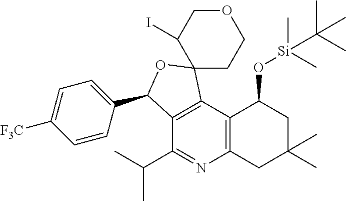 [SMILES: CC(C)c1nc2c(c3c1[C@@H](c1ccc(C(F)(F)F)cc1)OC31CCOCC1I)[C@@H](O[Si](C)(C)C(C)(C)C)CC(C)(C)C2]

To a solution of 34 g (R)-((S)-5-(tert-butyldimethylsilyloxy)-4-(3,6-dihydro-2H-pyran-4-yl)-2-isopropyl-7,7-dimethyl-5,6,7,8-tetrahydroquinolin-3-yl)(4-(trifluoromethyl)phenyl)methanol in 140 ml tetrahydrofurane and 280 ml acetonitrile are added 34 g lithium carbonate. After 10 min 68 g iodine are added and the reaction mixture is warmed to 50° C. and stirred for 70 h. 10 ml water are added and stirring is continued for 48 h at 60° C. and 30 min at 70° C. The reaction mixture is cooled to room temperature and diluted with 500 ml ethyl acetate. 200 ml 2M aqueous sodium thiosulfate solution are added and stirred for 40 min. The organic layer is separated and evaporated in vacuo. The residue is chromatographed twice on silica gel (cyclohexane/ethyl acetate 50:1).

Yield: 28.9 g (70% of theory)

Mass spectrometry (ESI⁺): m/z=716 [M+H]⁺

R_{f}-value: 0.53 (silica gel, cyclohexane/ethyl acetate 5:1)

### (3R,9S)-9-(tert-butyldimethylsilyloxy)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]

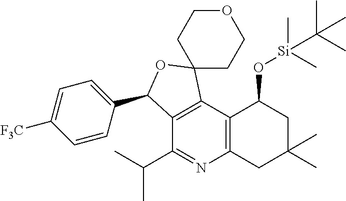 [SMILES: CC(C)c1nc2c(c3c1[C@@H](c1ccc(C(F)(F)F)cc1)OC31CCOCC1)[C@@H](O[Si](C)(C)C(C)(C)C)CC(C)(C)C2]

To a solution of 16 g (3R,9S)-9-(tert-butyldimethylsilyloxy)-3′-iodo-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran] in 250 ml methanol are added 8 ml triethylamine and 8 g palladium on charcoal (10%). After hydrogenation at 50 psi at room temperature for 2.5 h the reaction mixture is filtered. The filtrate is evaporated in vacuo and the residue is chromatographed on silica gel (cyclohexane/ethyl acetate 50:1.

Yield: 10.7 g (81% of theory)

Mass spectrometry (ESI⁺): m/z=590 [M+H]⁺

R_{f}-value: 0.47 (silica gel, cyclohexane/ethyl acetate 5:1)

### Final Compounds

### (3R,9S)-4-isopropyl-7,7-di methyl-3-(4-(trifluoromethyl)phenyl)-2′,3′,5′,6,6′,7,8,9-octahydro-3H-spiro[furo[3,4-c]quinoline-1,4′-pyran]-9-ol

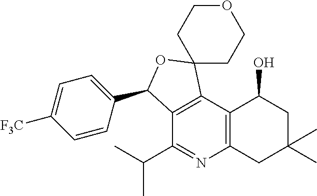 [SMILES: CC(C)c1nc2c(c3c1[C@@H](c1ccc(C(F)(F)F)cc1)OC31CCOCC1)[C@@H](O)CC(C)(C)C2]

To a solution of 12.5 g (3R,9S)-9-(tert-butyldimethylsilyloxy)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran] in 120 ml tetrahydrofurane are added 42 ml of a 1M tetrabutylammonium fluoride solution in tetrahydrofurane. After 17 h at room temperature the solvents are evaporated and the residue is dissolved in ethyl acetate and cyclohexane. The solution is washed several times with water and the organic layer is evaporated to dryness. The crude product is chromatographed on silica gel (cyclohexane/ethyl acetate 4:1). The solution is concentrated by evaporation of the solvents to obtain the title compound in crystalline free base form.

Yield: 9.2 g (91% of theory)

Mass spectrometry (ESI⁺): m/z=476 [M+H]⁺

R_{f}-value: 0.18 (silica gel, cyclohexane/ethyl acetate 5:1)

### Hydrogen Chloride Salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol

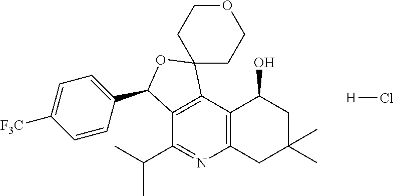 [SMILES: CC(C)c1nc2c(c3c1[C@@H](c1ccc(C(F)(F)F)cc1)OC31CCOCC1)[C@@H](O)CC(C)(C)C2 ; Cl]

To a solution of 400 mg (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol in 4 ml acetone are added 0.25 ml 4M HCl in dioxane. After 50 min the suspension is concentrated at 40° C. to approx. 2 ml. After 22 h at room temperature the suspension is filtered and the filter cake is dried in vacuo at 30° C. to obtain the crystalline title compound.

Yield: 405 mg (94% of theory)

Mass spectrometry (ESI⁺): m/z=476 [M+H]⁺ (free base)

### Hydrogen Bromide Salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol

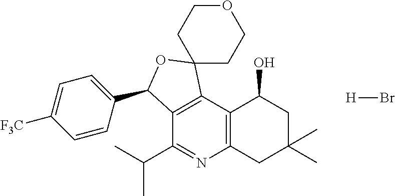 [SMILES: CC(C)c1nc2c(c3c1[C@@H](c1ccc(C(F)(F)F)cc1)OC31CCOCC1)[C@@H](O)CC(C)(C)C2 ; Br]

To a solution of 300 mg (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol in 1 ml acetone are added 85 μl aqueous HBr (48% in water). After 2 h 3 ml acetone are added and the solution is concentrated at 40° C. to approx. 1 ml. After 16 h at room temperature the resulting suspension is filtered and the filter cake is dried in vacuo at 30° C. to obtain the crystalline title compound.

Yield: 130 mg (37% of theory)

Mass spectrometry (ESI⁺): m/z=476 [M+H]⁺ (free base)

What is claimed is:

1. A crystalline hydrochloride salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

2. The crystalline hydrochloride salt according to claim 1, which is the monohydrochloride salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

3. The crystalline hydrochloride salt according to claim 1, which is in an anhydrous form.

4. The crystalline hydrochloride salt according to claim 1, which has an x-ray diffraction pattern substantially in accordance with that shown in FIG. 1.

5. The crystalline hydrochloride salt according to claim 1, characterised in that in the x-ray powder diagram it has characteristic values d=4.46 Å, 4.89 Å, 5.25 Å, 5.61 Å, 8.28 Å and 10.50 Å.

6. The crystalline hydrochloride salt according to claim 1, characterised by unit cell parameters approximately equal to the following:
a=30.13(1) Å,
b=14.64(1) Å,
c=6.068(4) Å,
α=β=γ=90°,
Volume=2677(3) Å³, and
Space group Pna2₁.

7. The crystalline hydrochloride salt according claim 1, which has a DSC and/or TG thermal curve substantially in accordance with that shown in FIG. 2.

8. The crystalline hydrochloride salt according to claim 7, which has a fusion temperature of about T_{fus}=220±10° C.

9. The crystalline hydrochloride salt according to claim 7, characterised in that its DSC profile shows a broad endothermic event between about 200 and about 250° C.

10. A process for making the crystalline hydrochloride salt of claim 1, the process comprising:
contacting a solution comprising the free base form of (3R,9S)-4-isopropyl-7,7-dimethyl-3-(4-(trifluoromethyl)phenyl)-2,3′,5′,6,6′,7,8,9-octahydro-3H-spiro[furo[3,4-c]quinoline-1,4′-pyran]-9-ol and acetone with HCl to form the hydrochloride salt of the compound of claim 1.

11. A crystalline hydrobromide salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

12. The crystalline hydrobromide salt according to claim 11, which is the monohydrobromide salt of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

13. The crystalline hydrobromide salt according to claim 11, which is in an anhydrous form.

14. The crystalline hydrobromide salt according to claim 11, which has a fusion temperature of about T_{fus}=230-232° C.

15. A process for making the crystalline hydrobromide salt of claim 11, the process comprising:
contacting a solution comprising the free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol and acetone with HBr to form the hydrobromide salt of the compound of claim 11.

16. A crystalline free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

17. The crystalline free base form according to claim 16, which is an anhydrous form.

18. The crystalline free base form according to claim 16, which has a fusion temperature of about T_{fus}=190-195° C. and which undergoes a reversible enantiotropic solid-state phase transition at about 55° C.

19. A method of making the crystalline free base form of the compound of claim 16, the process comprising:
forming a solution comprising the free base form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol and cyclohexane, and
crystallizing the crystalline free base of the compound of claim 16 from the solution.

20. Pharmaceutical composition comprising (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol as a crystalline free base form, a hydrochloride crystalline salt form or a hydrobromide salt form, optionally together with one or more inert carriers and/or diluents.

21. A method of using (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol as a crystalline free base form, a hydrochloride crystalline salt form or a hydrobromide salt form for treating or preventing diseases, disorders or conditions which can be influenced by inhibiting the cholesterol ester transfer protein (CETP), optionally in combination with one or more other therapeutic agents.

22. The method of claim 21, wherein the one or more other therapeutic agents comprises a statin.

23. A method for treating or preventing diseases, disorders or conditions which can be influenced by inhibiting the cholesterol ester transfer protein (CETP), the method comprising administering to a patient in need thereof a hydrochloride crystalline salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol.

24. A process for preparing a pharmaceutical composition comprising (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol as a crystalline free base form, a hydrochloride crystalline salt form or a hydrobromide crystalline salt form, the process comprising combining or mixing said free base form, said hydrochloride salt form, or said hydrobromide salt form of (3R,9S)-4-isopropyl-7,7-dimethyl-3-(4-(trifluoromethyl)phenyl)-2′,3′,5′,6,6′,7,8,9-octahydro-3H-spiro[furo[3,4-c]quinoline-1,4′-pyran]-9-ol, and one or more inert carriers and/or diluents.
